(19) United States
(12) Patent Application Publication
(10) Pub. No.: US 2022/0266004 A1
(43) Pub. Date: Aug. 25, 2022

(54) ATTENUATION OF MAGNETIC FIELD FOR CARDIAC IMPLANTABLE ELECTRONIC DEVICES

(71) Applicant: University of Florida Research Foundation, Inc., Gainesville, FL (US)

(72) Inventors: John N. Catanzaro, Jacksonville, FL (US); Christopher D. Batich, Gainesville, FL (US)

(21) Appl. No.: 17/676,442

(22) Filed: Feb. 21, 2022

Related U.S. Application Data
(60) Provisional application No. 63/151,168, filed on Feb. 19, 2021.

(51) Int. Cl. A61N 1/08; A61N 1/39; H05K 9/00

(52) U.S. Cl. CPC A61N 1/08; A61N 1/3925; H05K 9/009

(57) ABSTRACT

Disclosed are magnetic field shielding devices designed to attenuate magnetic fields that may interact with or otherwise have an adverse effect on implantable electronic devices. The magnetic field shielding devices may comprise a diverting or shielding material that is designed to shield implantable electronic devices from electromagnetic impulses and unwanted magnetic fields

### CROSS REFERENCE TO RELATED APPLICATION

This application claims benefit of U.S. Provisional Application No. 63/151,168 filed Feb. 19, 2021, entitled ATTENUATION OF MAGNETIC FIELD FOR CARDIAC IMPLANTABLE ELECTRONIC DEVICES, which is hereby incorporated by reference in its entirety for all purposes.

### BACKGROUND

Implantable cardiac defibrillators (ICDs) and pacemakers can comprise reed switches and/or sensors that are operated by an applied magnetic field. The reed switches and/or sensors are typically designed to trip at around 5-10 gauss magnetic field. Electronic devices that include embedded magnets (e.g., cell phones), as well as other types of magnetic devices (e.g., magnetic jewelry, magnetic name tags, etc.), have been known to “trip” or otherwise interact with the reed switch which can lead to dangerous effects on a patient's heart rhythm.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of an example magnetic field shielding device comprising a mobile device case and a shielding element according to various embodiments of the present disclosure.

FIG. 2A is a drawing of an example magnetic field shielding device comprising a mobile device case, a panel, and a shielding element according to various embodiments of the present disclosure.

FIG. 2B is a drawing of an example magnetic field shielding device comprising a mobile device case, a retractable panel, and a shielding element according to various embodiments of the present disclosure.

FIG. 3 is a drawing of an example magnetic field shielding device affixed to a portion of a body according to various embodiments of the present disclosure.

FIG. 4 is a drawing of an example magnetic field shielding device comprising a pouch containing a shielding element and implanted in a body according to various embodiments of the present disclosure.

FIGS. 5 and 5A illustrate drawings of an example magnetic field shielding device comprising a shielding element woven into a fabric of a shirt according to various embodiments of the present disclosure.

FIG. 6 is a drawing of an example magnetic field shielding device comprising a shielding element integrated into a vehicle according to various embodiments of the present disclosure.

FIG. 7 is a drawing of an example magnetic field shielding device comprising a shielding element integrated into pocket protector according to various embodiments of the present disclosure.

### DETAILED DESCRIPTION

Disclosed herein are various embodiments related to magnetic field shielding devices that are designed to attenuate magnetic fields that may interact with or otherwise have an adverse effect on implantable electronic devices. According to various embodiments, the magnetic field attenuation devices may comprise a diverting or shielding material that is designed to shield implantable electronic devices from electromagnetic impulses and unwanted magnetic fields.

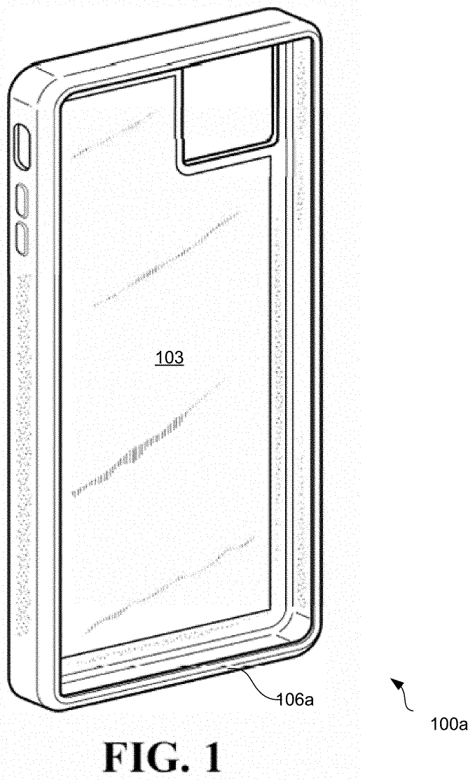

FIGS. 1-7 illustrate examples of a magnetic field shielding device 100 (e.g., 100a, 100b, 100c, 100d, 100e, 100f, 100g, 100h) according to various embodiments of the present disclosure. A magnetic field shielding device 100 may comprise a diverting or shielding element 103 that is in the form of a permalloy, foil, a mesh, a fiber, a powder, a coating, and/or other type of element as can be appreciated. In some examples, the diverting/shielding element may comprise a flexible sheet (e.g., plastic, elastomer, etc.), that comprises a metal powder, fiber and/or other forms as can be appreciated. In some examples, the shielding material may have a thickness of about 0.02 inches. In various examples, the shielding element comprises magnetically-permeable material such as, for example, a mu-metal alloy, a permalloy, a nanocrystalline grain structure ferromagnetic metal coatings, or other higher magnetically-permeable material that is capable of being fabricated into sheets, wires, or particles.

According to various examples, the shielding element may be integrated with an article that can be convenient to a person having an implantable electronic device, and can be used in combination with the shielding element to protect the user from any magnetic fields that may be emitted from magnetic or electronic devices having magnets that may interfere with the implantable electronic device. According to various examples, the article may comprise, for example, a mobile device case, apparel, an adhesive decal or patch, a pouch, a pocket protector, an automobile or vehicle, and/or other type of article as can be appreciated. The solutions provided herein offer two-dimensional magnetic shielding to minimize malfunctioning of implantable electronic devices due to magnetic field interference.

### Mobile Phone Case

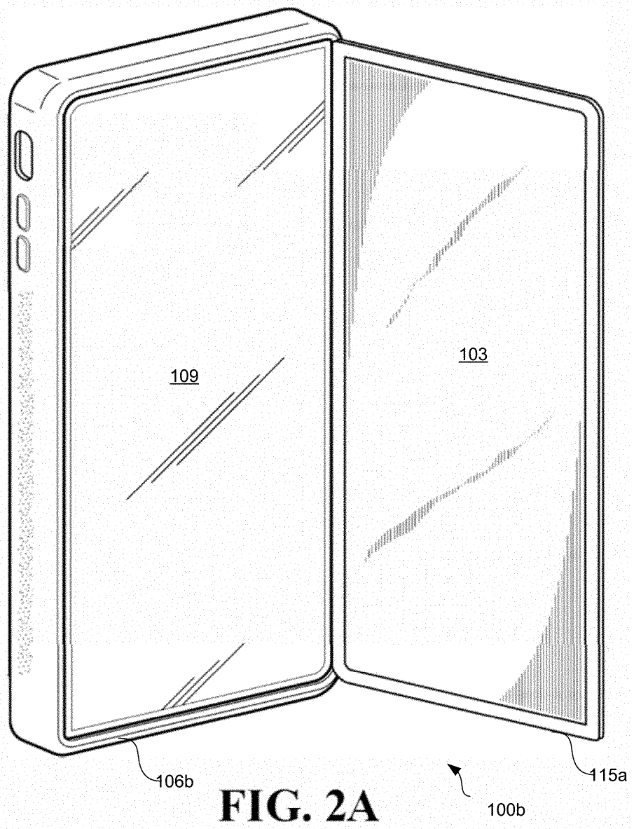

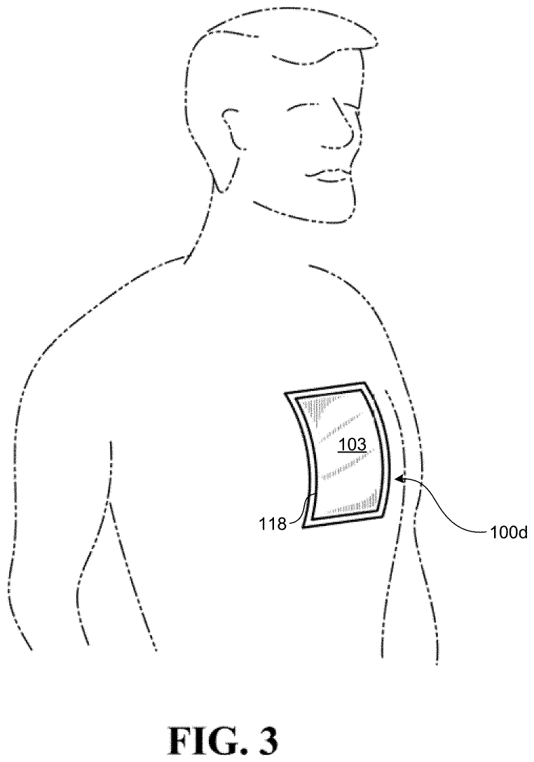

Turning now to FIGS. 1, 2A, and 2B shown are example drawings of a magnetic field shielding device 100a, 100b, 100c comprising the shielding element 103 integrated within a mobile device case 106 (e.g., 106a, 106b, 106c). Users may interact with mobile electronic devices 109 (FIGS. 2A-2B) (e.g., smartphones, cellular telephones, laptops, personal digital assistants, smartwatches, etc.) that may comprise magnets that can interfere with the operation of an implantable electronic device 112 (FIG. 4). By integrating the shielding element 103 within a mobile device case 106 that holds the mobile electronic device 109, the user with an implantable electronic device 112 may be protected from magnetic fields associated with the mobile electronic device 109.

In some examples, the shielding element 103 may comprise a foil or mesh that can be attached to the case 106. For example, the shielding element 103 may be attached to the case via an adhesive, hook and loop element, or other type of fastener.

In other examples, the shielding element 103 may be integrated into the case material. In particular, fibers can be made from spinnable polymers such as PET or polypropylene which contain micro or nano particles with magnetic field attenuation properties. The fibers can be then woven or spun into a pouch or case.

In some examples, as shown in FIGS. 2A and 2B, the case 106 (e.g., 106b, 106c) may comprise a panel component 115 (e.g., 115a, 115b) that may be “opened” or “closed” to expose the case material and/or mobile device 109. In this example, the panel component 115 may comprise the shielding element 103. When the user wishes to add the protection of the shielding element 103, the user may “close” the panel component 115 such that the panel component 115 covers at least a portion of the case or the mobile device 109 (e.g., a mobile device screen). In other examples, the user may “open” the panel component 115 to expose the at least a portion of the case or the mobile device 109 (e.g., a mobile device screen). In some examples, as illustrated in FIG. 2B, the panel component 115 may be retractable.

A user may wish to use the panel component 115 to provide protection when the mobile device 109 is not in use or is otherwise positioned about the user's body in a way that could cause interference with an implantable electronic device 112. For example, if the user places his or her mobile device 109 in a chest pocket of a shirt, the user may close the panel component 115 to minimize interference of the magnet in the mobile device 109 with the implantable electronic device 112.

### Sticker/Decal

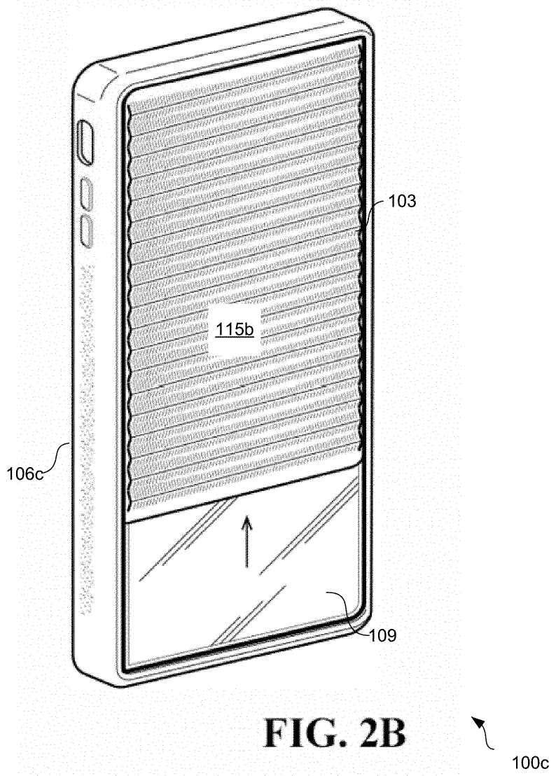

Moving on to FIG. 3 shown is an example drawing of a magnetic field shielding device 100d according to various examples. As illustrated in FIG. 3, the magnetic field shielding device 100d may comprise a shielding element 103 attached to an adhesive sheet 118 that comprises an adhesive material. For example, the shielding element 103 may be coupled to an adhesive sheet 118 comprising, for example, an adhesive decal, patch, or sticker, that can be attached to a user or an article of clothing that can be worn by a user. The magnetic field shielding device 100 may be placed on the user or the article of clothing worn by the user in a position that places the shielding element 100 over the implanted electronic device 112.

For example, the user having an implantable electronic device 112 that is implanted in his or her heart (e.g., pacemaker, defibrillator, etc.) may place the adhesive decal 118 comprising the shielding element 103 over the user's chest such that the shielding element 103 blocks any electromagnetic fields that may be emitted by electronic devices or magnets that may be in close proximity to the implantable electronic device 112. In some examples, the magnetic field shielding device 100 may comprise a hook and loop fastener. In this examples, the shielding element 103 may be coupled to a hook portion of a loop portion of the hook and loop fastener, as can be appreciated. In addition, a decal or adhesive of the attachment 118 may be embodied to stick directly onto the skin or apparel.

### Pouch

Turning now to FIG. 4, shown is a drawing of an example of a magnetic field shielding device 100e comprising a pouch device 121 comprising the shielding element 103 according to various examples. In another embodiment of the present disclosure, the magnetic field shielding device 100 may comprise a pouch device 121 including the shielding element 103. For example, the shielding element 103 may comprise a magnetically permeable powder, or other type of material, that is placed within the pouch device 121. The pouch device 121 may comprise a flexible material including, for example, a plastic, that comprises one or more panels joined to one another to comprise an opening that can be filled with the shielding element 103.

In some examples, as shown in FIG. 4, the magnetic field shielding device 100 comprising the pouch device 121 may be implantable in proximity to the implantable electronic device 112 of the user. In this example, the pouch may be configured to be implantable over or otherwise in proximity to the implantable electronic device 112 in whole or multiple parts to allow for battery-changing surgery associated with the implantable device 112 between the parts.

In some examples, magnetic shielding device 100 may comprise a pouch device 121 including particles or fibers of the shielding material 03 that are embedded in a plastic or rubbery matrix and are available as a spun pouch or a solid film that can be used to manufacture the pouch device 121.

### Apparel

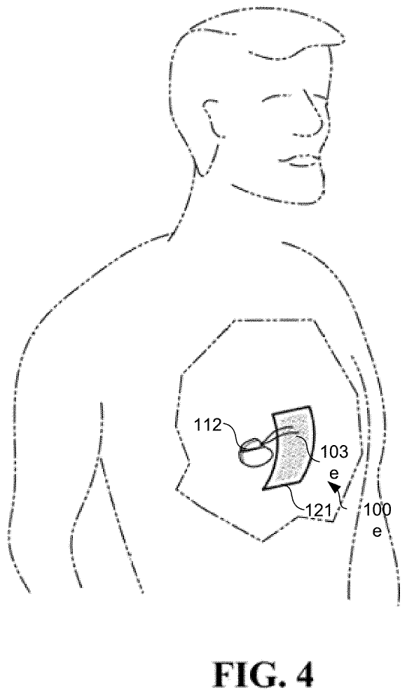

Moving on to FIGS. 5 and 5A, shown are drawings of a magnetic field shielding device 100f comprises an article of clothing 124 comprising the shielding element 103. In particular, the shielding element 103 may comprise a mesh or fiber that is woven with a textile material 127. In this example, an article of clothing 124 may be manufactured with the shielding element 103 woven into the textile material 127 of the article of clothing 124 to provide protection against magnetic fields that may interfere with an implantable electronic device 112. The weave associated with the shielding element 103 and the textile material 127 comprise a weave design that provides a suitable amount of the shielding element 103 integrated with the textile material 127 such that the shielding element can deflect or attenuate flux density from a magnetic field.

In various examples, the shielding element 103 may be woven into the textile material 127 in areas associated with the article of clothing 124 that may be positioned over the implantable electronic device 112. In the example of a pacemaker or defibrillator, the area of a shirt that is placed over a person's chest, may comprise an area or patch of textile material 127 that is woven with the shielding element 103, as shown in FIG. 5.

In various examples, the shielding element fibers 103 can be combined with the regular textile fibers 127 to make mixed multifilament bundles, or special fibers by themselves. All can be made by wet, dry, or melt spinning techniques.

### Vehicle

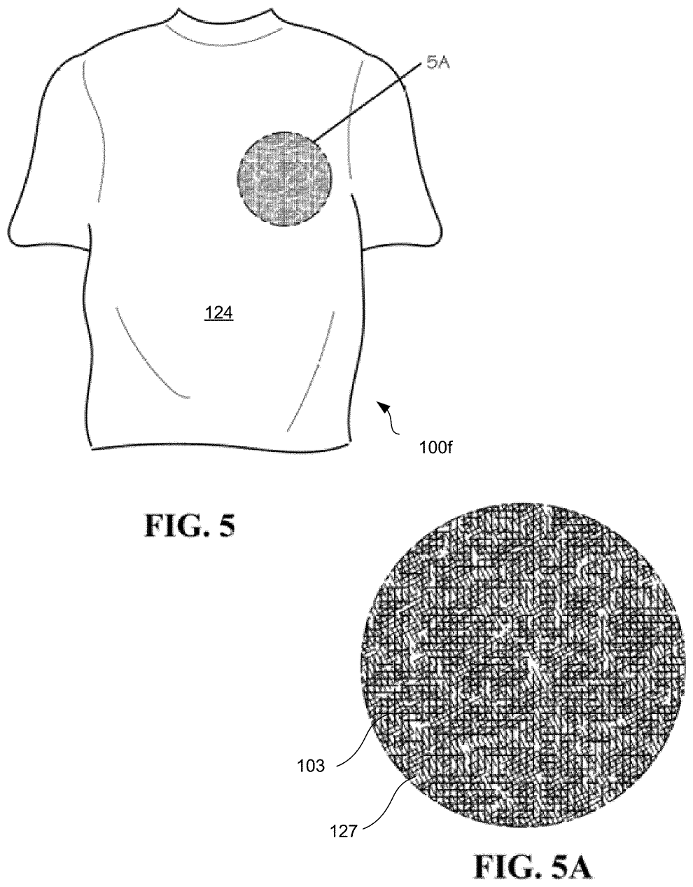

FIG. 6 illustrate a drawing of an example magnetic field shielding device 100g according to various examples. For example, the magnetic field shielding device 100g may comprise a vehicle 130 (e.g., car, airplane, train, helicopters, spaceships, military vehicles, etc.) comprising the shielding element 103. For example, a vehicle 1006 may comprise a panel, a coating, a decal, or other element as can be appreciated, that comprises the shielding element 103. Therefore, an individual associated with the vehicle (e.g., driver, passenger) who has an implantable electronic device 112 may be protected from electromagnetic fields associated with charging stations for electric vehicles or other means of magnetic field exposure related to a vehicle 130. In addition, the shielding element 103 may be used for defense purposes against any potential attacks using magnetic fields. This material may also be included as an inner liner of the vehicle chassis, floor mats, seat covers, or other portion of the vehicle 130, as can be appreciated.

### Pocket Protector

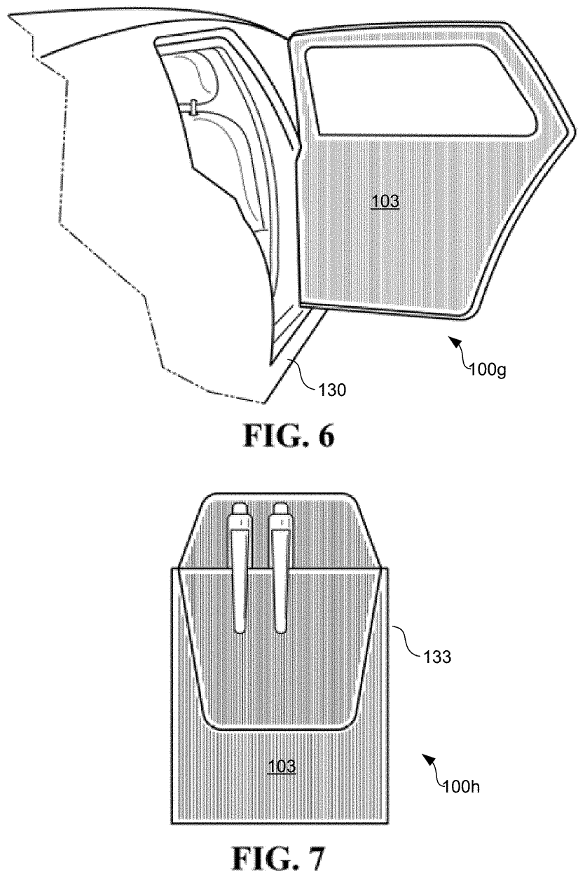

Turning now to FIG. 7, shown is an example drawing of a magnetic field shielding device 100h comprising a pocket protector 133 that may be inserted into a shirt pocket to hold writing instruments and/or other objects in the shirt pocket. In particular, the magnetic field shielding device 100 may comprise a protector panel and a shielding element 103 that is integrated within the protector panel. In some examples, the pocket protector 133 may comprise an impregnated clip for a shirt pocket that comprises a covering and/or coating that includes the shielding element.

Hardware (Inside) of an Implantable Cardioverter Defibrillator or Pacemaker or around the Accompanying Transvenous, Subcutaneous or Epicardial Leads/Coils.

In another embodiment, the shielding element 103 comprising the mu metal or permalloy described herein, may be incorporated into the hardware of an implantable electronic device 112 (e.g., pacemaker or defibrillator) including transvenous or subcutaneous implantable cardioverter pulse generators or the cardiac leads and or coils which are implanted.

In some examples, a sequence of events may be required to engage the reed switch or Hall effect sensor to access the implantable electronic device 112. For example, a sequence of events may include an additional contact with a magnet and an orientation identified by the implantable electronic device 112 that is similar to the orientation required for a mobile device engagement to enter.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. An apparatus for attenuating magnetic fields that interfere with an implantable electronic device, comprising:
an item comprising one of a mobile device case, a sealed pouch, an adhesive sheet, a vehicle, an implantable electronic device, a pocket protector, or an article of clothing; and
a shielding element integrated within the item, the shielding element being configured to deflect or attenuate a flux density from the magnetic field of a magnet.

2. The apparatus of claim 1, wherein the shielding element comprises at least one of a foil, a mesh, a fiber, a powder, or a coating.

3. The apparatus of claim 1, wherein a material of the shielding element comprises a mu-metal alloy or a permalloy.

4. The apparatus of claim 1, wherein the item comprises the mobile device case.

5. The apparatus of claim 4, wherein the shielding element is attached to the mobile device case via an adhesive.

6. The apparatus of claim 4, wherein the mobile device case comprises a panel component configured to cover or uncover a portion of the mobile device case.

7. The apparatus of claim 6, wherein the shielding element being integrated within a material of the panel component.

8. The apparatus of claim 6, wherein the panel component comprises an adhesive attached to a face of the panel component, the shielding element being integrated within the adhesive.

9. The apparatus of claim 6, wherein the shielding element comprises a coating, the panel component being coated with the coating.

10. The apparatus of claim 6, wherein the panel component is retractable.

11. The apparatus of claim 1, wherein the item comprises the adhesive sheet comprising an adhesive material on a first side of the adhesive sheet, and the shielding element is disposed about a second side of the adhesive sheet.

12. The apparatus of claim 11, wherein the adhesive sheet is configured to adhere to at least one of skin or an article of clothing worn by a person having the implantable electronic device.

13. The apparatus of claim 1, wherein the item comprises the sealed pouch and the shielding element is disposed within an interior of the sealed pouch.

14. The apparatus of claim 13, wherein the sealed pouch is configured to be implanted in proximity to the implanted electronic device of a person.

15. The apparatus of claim 1, wherein the item comprises the article of clothing comprising a textile material and the shielding element being woven with the textile material in a weave pattern designed to deflect or attenuate a flux density from the magnetic field of a magnet.

16. The apparatus of claim 1, wherein the item comprises the vehicle, and the shielding element is disposed about the vehicle, the shielding element being positioned to the vehicle at a position of the vehicle that protects an individual associated with the vehicle and having the implantable electronic device from magnetic fields associated with a magnetic device that is within a proximate range to the vehicle.

17. The apparatus of claim 16, wherein the vehicle comprises a car, an airplane, a train, a helicopter, a spaceship, or a military vehicle.

18. The apparatus of claim 1, wherein the item comprises the pocket protector, the shielding element being integrated within the pocket protector, and the shielding element is configured to deflect or attenuate magnetic fields associated with a magnetic device from the implantable electronic device of a person wearing the pocket protector.

19. The apparatus of claim 1, wherein the shielding element has a thickness of about 0.02 inches.

20. A magnetic field shielding device for attenuating magnetic fields that interfere with implantable electronic device, comprising:
an item comprising a mobile device case, a sealed pouch, an adhesive sheet, a vehicle, an implantable electronic device, a pocket protector, or an article of clothing; and
a shielding element integrated within the item, the shielding element being configured to attenuate magnetic fields, the shielding element comprises at least one of a foil, a mesh, a fiber, a powder, or a coating, and a material of the shielding element comprising a mu-metal alloy or a permalloy.
